(19) United States
(12) Patent Application Publication
(10) Pub. No.: US 2026/0337425 A1
(43) Pub. Date: Oct. 29, 2026

(54) Modified Sophorolipids with Enhanced Dispersion Properties

(71) Applicant: Locus Solutions IPCo, LLC, Solon, OH (US)

(72) Inventors: Lee SPEIGHT, Solon, OH (US); Andrew MORRIS, Richmond, VA (US); Daniel HAGAMAN, Mechanicsville, VA (US)

(21) Appl. No.: 19/109,023

(22) PCT Filed: Sep. 18, 2023
(86) PCT No.: PCT/US2023/074491

§ 371 Date: Mar. 5, 2025

Related U.S. Application Data
(60) Provisional application No. 63/407,759, filed on Sep. 19, 2022.

(51) Int. Cl. C09D 7/63; C07H 17/08; C09D 5/14; C09D 7/61

(52) U.S. Cl. CPC C09D 7/63; C07H 17/08; C09D 5/14; C09D 7/61

(57) ABSTRACT

This present invention relates to modified sophorolipid molecules for enhancing the dispersion of particulate substances, including nanoparticles. In certain embodiments, the compositions and methods can be used for improved dispersion in paint and coating formulations and for improved dispersion of mined materials.

### CROSS-REFERENCE TO RELATED APPLICATION

This application claims priority to U.S. Provisional Application No. 63/407,759, filed Sep. 19, 2022, which is incorporated herein by reference in its entirety.

### BACKGROUND OF THE INVENTION

A nanoparticle is a small particle that is typically characterized as being between 1 to 100 nanometers in size. Nanoparticles, in general, are a large family of both organic and inorganic materials. Each material has uniquely tunable properties and thus can be selectively designed for specific applications. Nanoparticles are undetectable by the human eye, and due to their small size, have a large surface area to volume ratio when compared to bulk material, such as powders, plates and sheets. Properties such as cation exchange capacity, enhanced diffusion, ion adsorption and complexation are: enhanced at the nanoscale.

The use of nanomaterials spans across a wide variety of industries, from healthcare and cosmetics, to environmental preservation and materials science. For example, mineral nanoparticles, such as titanium oxide, can be used in sunscreens as UV protection agents with enhanced stability. Nanoparticles can also be used for creating liposome, or nanocapsule, drug delivery systems.

One industry that benefits from nanoparticle technology is the paint and primer industry. A paint primer is comprised of a mixture of pigments, binders and solvents, or carriers. Ideally, the pigments are evenly dispersed throughout the formulation so that only a thin layer of primer is required to adequately cover a surface.

A universal architectural primer coating serves two main functions. The first is to enhance the ability of a topcoat to beautify the substrate. The second main function is to enhance the durability, extending the life of the topcoat. These functions are primarily achieved by improving adhesion, which can lead to better crack and blister resistance. Additionally, critical performance needs include hiding topographic variations, sealing porous substrates, preventing old colors from showing through the topcoat, odor blocking, providing a tie-coat layer between the surface and topcoat, and reducing stains from migrating into the topcoat.

Examples of nanoparticles used in paints and coatings are titanium dioxide (TiO₂), silver (Ag), and silicon dioxide (SiO₂). The photocatalytic and hydrophobic properties of titanium dioxide nanoparticles provides for coatings with self-cleaning, air purifying, and anti-UV properties. Nano-silver can have antimicrobial properties, whereas silicon dioxide nanoparticles can increase the scratch- and fire-resistance of paints and coatings.

Zinc nanoparticles are also utilized in primers as undercoats to protect steel surfaces from corrosion in the automotive, marine, chemical plant, oil and gas, industrial machinery and construction industries. Unlike regular paints or epoxies, which resist corrosion by forming impermeable barriers between the metal and atmospheric moisture, zinc-rich primers provide additional corrosion protection by electrical means. If the paint is scratched during its operational life, the zinc will sacrificially corrode rather than the steel.

Another industry that benefits from nanoparticles is the oil and gas industry. Adding certain nanoparticles, such as TiO₂, CuO, silica-, alumin- and/or carbon-based nanoparticles, to injection solutions can significantly improve enhanced oil recovery (EOR) techniques, with advantages such as wettability alteration, changes in fluid properties, improving mobility of trapped oil, enhancing the consolidation of sands and decreasing the interfacial tension (IFT) in a reservoir. Nanoparticle fluid systems can also be designed for remediation of paraffin and asphaltene deposits, polymers, biofilms and scale, as well as for reducing the viscosity of bitumen and heavy oils.

Another industry that benefits from nanoparticle technology is the mining industry. When minerals are effectively dispersed in water, the mineral fluids are less viscous and therefore easier to handle, pump, grind, float, and transport.

While nanoparticles have myriad benefits and uses, the formation of solutions having uniform dispersions of nanoparticles remains a challenge. Nanoparticles often form clusters, called aggregates and agglomerates due to, for example, their electrostatic properties. The strength of the bonds can vary, ranging from covalent bonds to weak electrostatic or magnetic forces. For optimum efficacy of nanoparticulate compositions, even distribution of nanoparticles with reduced clustering is ideal. Chemical reagents may be used to force particles to separate; however, more environmentally-friendly methods are desired.

Biosurfactants are chemicals that are part of a growing trend of using microbially-sourced chemicals to replace synthetic chemicals. Biosurfactants are a structurally diverse group of surface-active substances produced by microorganisms. All biosurfactants are amphiphiles. They consist of two parts: a polar (hydrophilic) moiety and non-polar (hydrophobic) group. Due to their amphiphilic structure, biosurfactants can, for example, increase the surface area of hydrophobic water-insoluble substances, increase the water bioavailability of such substances, and reduce interfacial tension at interfaces. Accordingly, because of their environmentally-friendly nature and their wide-ranging uses, biosurfactants have the potential to replace chemicals in a variety of applications and industries.

### BRIEF SUMMARY OF THE INVENTION

The subject invention provides materials and methods for improving the dispersion of substances, including particulate substances and nanoparticles, in compositions utilized in various industries. In some embodiments, these applications include, for example, paint and coatings, oil and gas recovery, mining, water treatment and agriculture.

In preferred embodiments, the subject invention provides modified sophorolipids having dispersion-promoting properties. The subject invention also provides compositions comprising such modified sophorolipids as dispersion agents. The subject invention further provides materials and methods for producing sophorolipids that are amenable to modification; and materials and methods for modifying sophorolipids to create biobased dispersion agents.

In one embodiment, the subject invention provides a compound that can be used as a dispersion agent. In preferred embodiments compounds of the subject invention having dispersion properties, have a structure according to, for example, General Formula (II) or (IV):

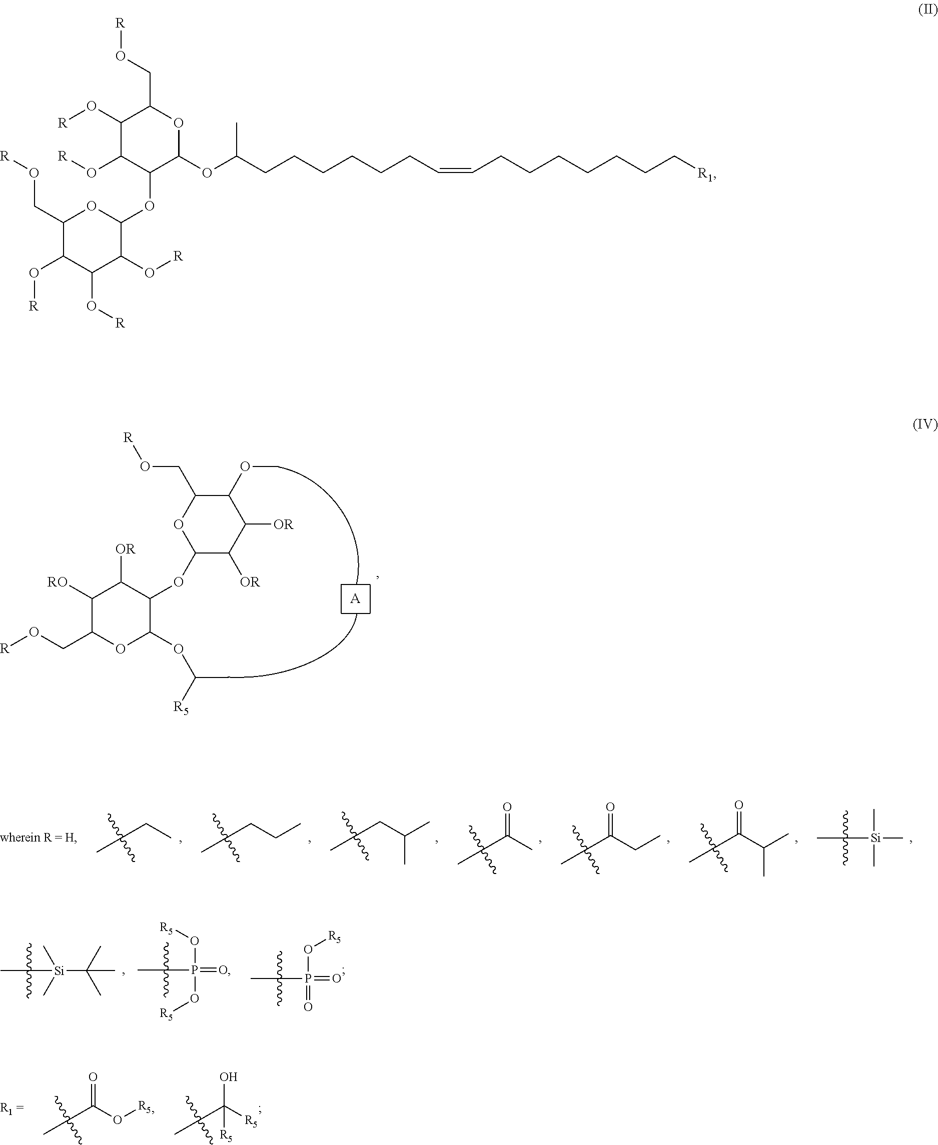

R₂═CH₂, (CH₂)₂, (CH₂)₃, (CH₂)₄, (CH₂)₅, (CH₂)₆, (CH₂)₇, (CH₂)₈, (CH₂)₉ and (CH₂)₁₀;
R₃═H, methyl, ethyl, propyl, butyl, pentyl, i-propyl, i-butyl,

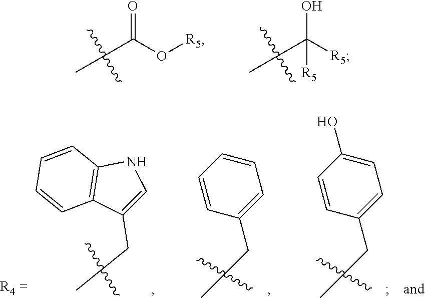

R₅═H, methyl, ethyl, propyl, butyl, pentyl, i-propyl, i-butyl,

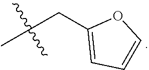 [SMILES: *Cc1ccco1]

In preferred embodiments, the aliphatic chain A has 10 to 21 carbons such that the total number of carbons in the aliphatic chain A, R₅, and the carbon to which R₅ is attached is 12 to 22 carbons. In some embodiments, A is an unsaturated aliphatic chain having 16 or 17 carbons with one, two, or three unsaturations. In some embodiments, A is a fully saturated aliphatic chain.

In one embodiment, the subject invention provides a method for dispersing a particulate substance in a liquid carrier, the method comprising contacting a dispersion agent with the particulate substance concurrently with, before or after mixing the particulate substance with the liquid carrier, wherein the dispersion agent comprises one or more of the modified sophorolipids of the present invention. The liquid may be an aqueous or non-aqueous fluid, including water, an alcohol, and/or an oil.

The particulate substance can comprise particles ranging from, for example, 0.1 nm to 1 cm in size, for example, a pigment or dye particle, a metallic particle, an organic particle, a polymeric particle, a liposomal particle, or others. In some embodiments, the particulate substance is a nanoparticle having a particle size of about 1 to 100 nm.

In one embodiment, the subject invention also provides a method for preventing or reducing the settling of a dispersed particulate substance in a liquid, wherein the method comprises applying the dispersion agent of the subject invention to particulate substance and/or the liquid.

In a specific embodiment, the method hinders the agglomeration of particles and/or promotes the separation and/or distribution of particles evenly throughout the liquid.

In certain embodiments, the subject composition can be applied to a colloid, emulsion, mineral slurry, or mineral flotation mixture. In certain embodiments, the subject composition can be applied to the vessel that contains the colloid, emulsion, mineral slurry, or mineral flotation mixture.

In certain embodiments, the subject composition can be used to separate agglomerated particles in a mineral flotation or in mining wastewater.

Further provided herein are dispersion formulations comprising a dispersion agent according to the subject invention and a particulate substance in a carrier. The formulation can be, for example, a colorant or coating, for example, a paint, primer, ink, dye or waterproofing treatment. These formulations can be useful in, for example, home improvement, textile and papermaking, and for covering equipment, building materials, and automobiles.

### DETAILED DESCRIPTION OF THE INVENTION

The subject invention provides biobased dispersion agents, and compositions comprising the biobased dispersion agent for household and industrial uses. Advantageously, the biobased dispersion agent can promote the dispersion of particulate substances in a liquid and/or prevent the agglomeration and/or settling out of particles. Preferably, the biobased dispersion agent is biosurfactant, more preferably, a sophorolipid.

The subject invention also provides dispersion formulations comprising a particulate substance and a biosurfactant for use in various applications, for example, in paints and coatings, agriculture, oil and gas, mining operations, and water treatment.

### SELECTED DEFINITIONS

As used herein, enhancing the “dispersion” of a particulate substance means promoting a substantially even or uniform distribution of particles suspended throughout a fluid. Promoting a substantially even distribution means reducing the degree to which the particles cluster together into agglomerates and/or aggregates and/or reducing the size and/or number of such clusters. An agglomerate is a reversible collection of particles that are weakly bound by, for example, van der Waals forces, whereas an aggregate is comprised of particles held together by stronger covalent bonds.

In some embodiments, the degree of dispersion of the nanoparticles in a composition produced according to the subject invention can be measured by, for example, the zeta potential, where suspensions possessing a high absolute value of zeta potential are considered “well-dispersed.” See Fairhurst 2013, incorporated by reference herein.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An isolated microbial strain means that the strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 98%, by weight the compound of interest. For example, a purified compound is one that is at least 80%, 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism or a substance necessary for taking part in a particular metabolic process. A metabolite can be an organic compound that is a starting material, an intermediate in, or an end product of metabolism. Examples of metabolites include, but are not limited to, enzymes, acids, solvents, alcohols, proteins, vitamins, minerals, microelements, amino acids, biopolymers and biosurfactants.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

As used herein, the term “alkyl” refers to straight chain or branched hydrocarbon groups. Suitable alkyl groups include, but are not limited to methyl, ethyl, propyl, isopropyl, butyl, isobutyl, pentyl, hexyl, heptyl, octyl, nonyl, decyl, dodecyl, tetradecyl, hexadecyl and octadecyl. The term alkyl may be prefixed by a specified number of carbon atoms to indicate the number of carbon atoms or a range of numbers of carbon atoms that may be present in the alkyl group such as C1-C10 alkyl, C1-C20 alkyl, and C10-C20 alkyl. For example, C1-C3 alkyl refers to methyl, ethyl, propyl and isopropyl.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

As used herein, “surfactant” refers to a substance or compound that reduces surface tension when dissolved in water or water solutions, or that reduces interfacial tension between two liquids, or between a liquid and a solid. The term “surfactant” thus includes cationic, anionic, nonionic, zwitterionic, amphoteric agents and/or combinations thereof.

“Biosurfactants” are microbially-derived amphiphilic molecules consisting of both hydrophobic (e.g., a fatty acid) and hydrophilic domains (e.g., a sugar). Use of biomolecules classified as biosurfactants is attractive for industry as a means for combining green chemistry with a low carbon footprint.

Sophorolipids, in particular, are glycolipid biosurfactants produced by, for example, various yeasts of the Starmerella clade. Sophorolipids comprise a sophorose consisting of two glucose molecules, linked to a fatty acid by a glycosidic ether bond. Sophorolipids are categorized into two general forms: the lactonic form, where the carboxyl group in the fatty acid side chain and the sophorose moiety form a cyclic ester bond; and the acidic form, or linear form, where the ester bond is hydrolyzed.

Sophorolipids consist of a disaccharide sophorose linked to long chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid can have, for example, 11 to 20 carbon atoms, and may contain one or more unsaturated bonds.

Furthermore, the sophorose residue can be acetylated on the 6- and/or 6′-position(s). The fatty acid carboxyl group can be free (acidic or linear form) or internally esterified at the 4″-position (lactonic form). In most cases, fermentation of sophorolipids results in a mixture of hydrophobic (water-insoluble) sophorolipids, including, e.g., lactonic sophorolipids, mono-acetylated linear sophorolipids and di-acetylated linear sophorolipids, and hydrophilic (water-soluble) sophorolipids, including, e.g., non-acetylated linear sophorolipids.

Sophorolipids are frequently produced as a mixture of related molecules. Differences among the related molecules arise mainly from: their fatty acid structure (degree of unsaturation, chain length, position(s) of unsaturation and position of hydroxylation); whether they are produced in the linear or lactonic form; the acetylation pattern; the presence of stereoisomers; and/or whether the glycosidic bond on the fatty acid is at the ω-position (e.g., terminal) or ω-1 position (sub-terminal).

The sophorolipid compounds of the subject invention have many advantageous characteristics that make them superior to synthetic surfactants, such as biodegradability, low toxicity, high surface and interfacial activities, and stability under wide ranges of temperatures, pressures, and ionic strengths. On the other hand, functional properties of sophorolipids can differ between lactonic and acidic forms. For example, acidic sophorolipids generally have higher hydrophile-lipophile balance (HLB) than lactonic sophorolipids, while lactonic sophorolipids generally have lower HLB and greater surface tension-reducing properties than acidic sophorolipids. Additionally, acidic sophorolipids are typically highly water soluble due to their free carboxylic acid groups.

As used herein, the term “sophorolipid,” and “sophorolipid molecule” includes all forms, and isomers thereof, of sophorolipid molecules, including, for example, acidic (linear) sophorolipids and lactonic sophorolipids. Further included are mono-acetylated sophorolipids, di-acetylated sophorolipids, esterified sophorolipids, sophorolipids with varying hydrophobic chain lengths, sophorolipids with fatty acid-amino acid complexes attached, and other, including those that are and/or are not described within in this disclosure. In certain embodiments, the subject compositions can comprise lactonic and linear SLP, with at least about 50%, about 55%, about 60%, about 65%, about 70%, about 75%, about 80%, about 85%, about 90%, or about 95% of the SLP comprising linear forms, and the remainder comprising lactonic forms.

### Modified Sophorolipids

Linear sophorolipid molecules are typically represented by General Formula (I):

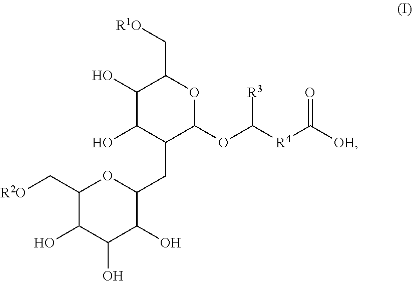

wherein R¹ and R² are each independently a hydrogen or an acetyl group; R³ is hydrogen or an alkyl group; and R⁴ is a saturated aliphatic hydrocarbon chain, or an unsaturated aliphatic hydrocarbon chain having at least one double bond, and may have one or more Substituents.

Examples of the Substituents can include halogen atoms, hydroxyl, lower (C1-6)alkyl groups, halo lower (C1-6)alkyl groups, hydroxy lower (C1-6)alkyl groups, halo lower (C1-6)alkoxy groups, and others. Sophorolipid molecules can be obtained as a collection of 30 or more molecules having different fatty acid chain lengths and degrees of unsaturation (R⁴). R⁴ typically has 11 to 20 carbon atoms. In preferred embodiments of the subject invention, R⁴ has 16-17 carbon atoms.

In certain embodiments, the present invention provides modified sophorolipids. In some embodiments, the modification includes, for example, peracetylation and/or esterification. In one embodiment, modification includes, for example, a sophorolipid or a truncated sophorolipid linked to an amino acid via a primary amine. By “truncated,” it is meant that the carbon chain of the fatty acid is shorter than the carbon chain of the fatty acids normally present in sophorolipids.

In certain embodiments, the modified sophorolipid of the subject invention has a structure of General Formula (II):

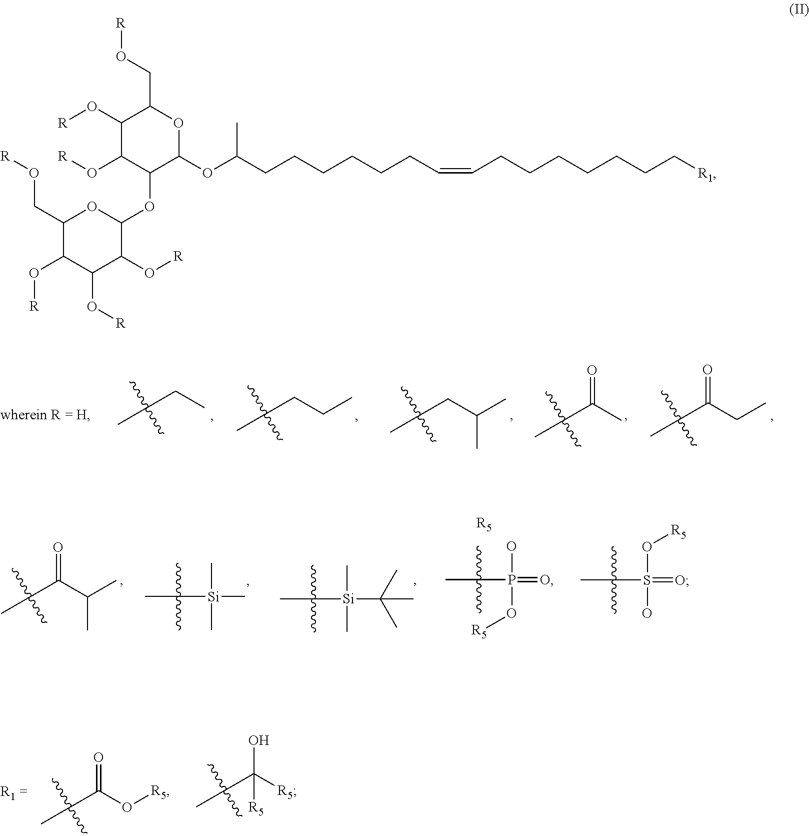

and R₅═H, methyl, ethyl, propyl, butyl, pentyl, i-propyl, i-butyl,

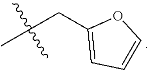 [SMILES: *Cc1ccco1]

In one embodiment, the present invention provides modified sophorolipids of General Formula (III):

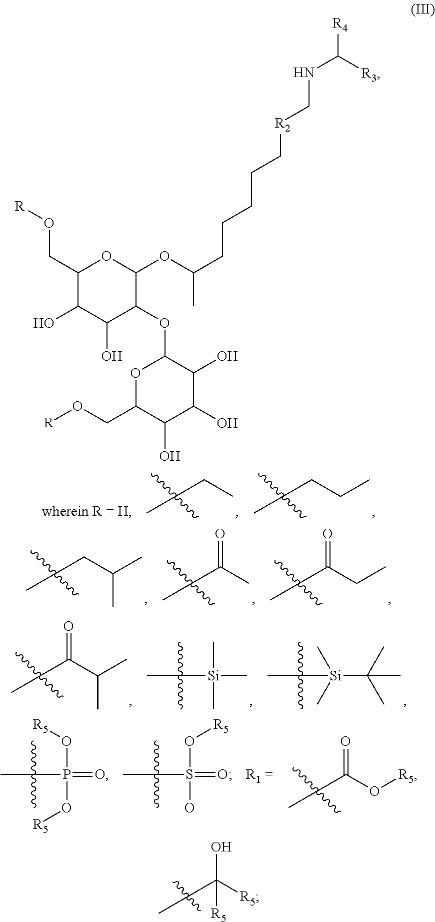

R₂═CH₂, (CH₂)₂, (CH₂)₃, (CH₂)₄, (CH₂)₅, (CH₂)₆, (CH₂)₇, (CH₂)₈, (CH₂)₉ and (CH₂)₁₀; R₃═H, methyl, ethyl, propyl, butyl, pentyl, i-propyl, i-butyl,

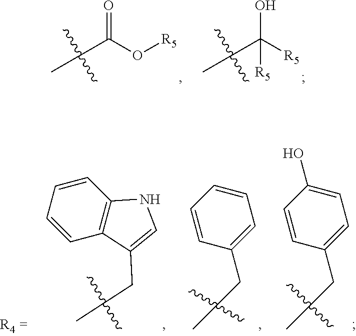

and R₅═H, methyl, ethyl, propyl, butyl, pentyl, i-propyl, i-butyl,

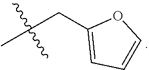 [SMILES: *Cc1ccco1]

In one embodiment, the present invention provides modified sophorolipids having General Formula (IV), which are cyclic ether analogs of lactonic sophorolipids:

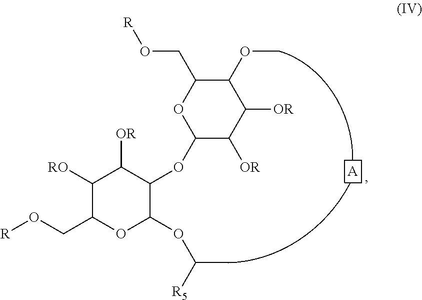

wherein A=a saturated or unsaturated aliphatic chain that is optionally substituted;

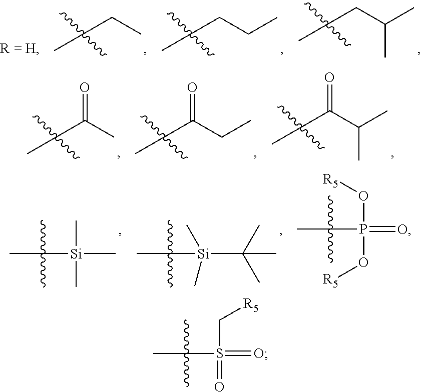

and R₅═H, methyl, ethyl, propyl, butyl, pentyl, i-propyl, i-butyl,

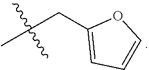 [SMILES: *Cc1ccco1]

In preferred embodiments, the aliphatic chain A has 10 to 21 carbons such that the total number of carbons in the aliphatic chain A, R₅, and the carbon to which R₅ is attached is 12 to 22 carbons. In some embodiments, A is an unsaturated aliphatic chain having 16 or 17 carbons with one, two, or three unsaturations. In some embodiments, A is a fully saturated aliphatic chain.

In one embodiment, the present invention provides modified sophorolipids having General Formula (V):

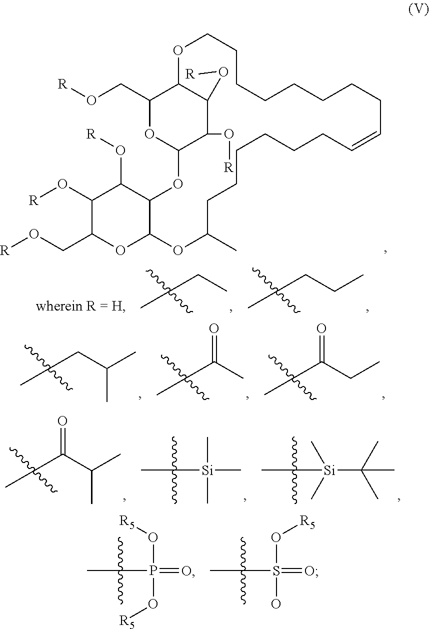

and R₅═H, methyl, ethyl, propyl, butyl, pentyl, i-propyl, i-butyl,

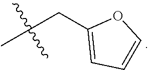 [SMILES: *Cc1ccco1]

In one embodiment, the present invention provides modified sophorolipids having the general formula (VI):

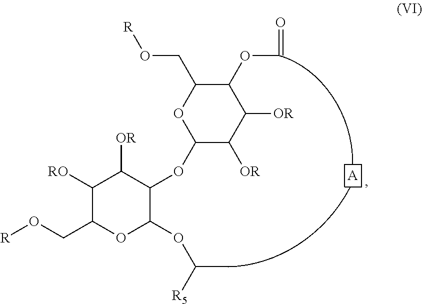

wherein A=a saturated or unsaturated aliphatic chain that is optionally substituted;

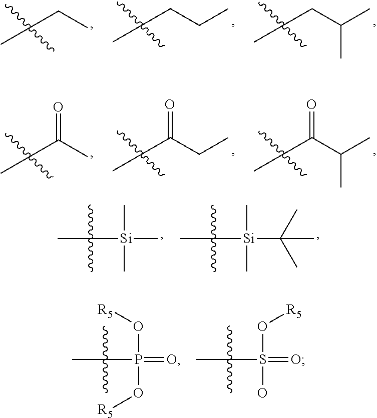

and R₅═H, methyl, ethyl, propyl, butyl, pentyl, i-propyl, i-butyl,

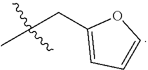 [SMILES: *Cc1ccco1]

In preferred embodiments, the aliphatic chain A has 10 to 21 carbons such that the total number of carbons in the aliphatic chain A, R₅, and the carbon to which R₅ is attached is 12 to 22 carbons. In some embodiments, A is an unsaturated aliphatic chain having 16 or 17 carbons with one, two, or three unsaturations. In some embodiments, A is a fully saturated aliphatic chain.

In one embodiment, the present invention provides modified sophorolipids having the general formula (VII):

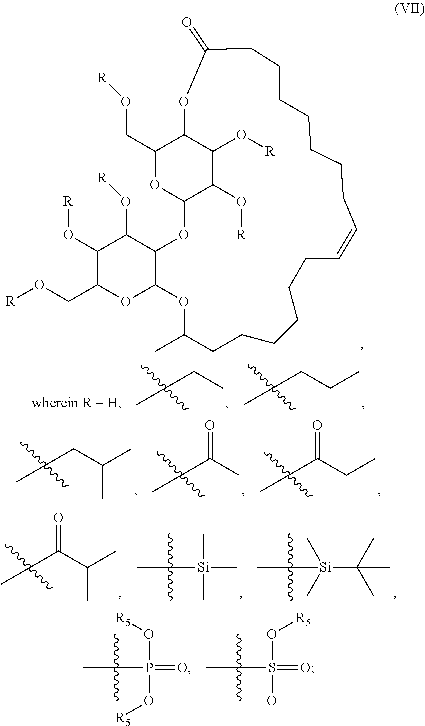

and R₅═H, methyl, ethyl, propyl, butyl, pentyl, i-propyl, i-butyl,

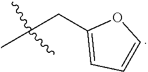 [SMILES: *Cc1ccco1]

In one embodiment, the modified sophorolipids of the present invention have a reduced HLB value compared to unmodified linear or lactonic sophorolipids. In specific embodiments, the modified sophorolipids of the present invention have a HLB value equal to or less than 6, preferably, equal to or less than 5, more preferably, equal to or less than 4, most preferably, equal to or less than 3.

### Production of Sophorolipids

The subject invention provides materials and methods for producing and modifying sophorolipids. Advantageously, the subject invention is suitable for industrial scale production of purified sophorolipids and uses safe and environmentally-friendly materials and processes.

Sophorolipids are generally obtained from fermentation by microorganisms that use as carbon sources pure fatty acids, fatty acid mixtures, pure fatty acid esters, mixtures of fatty acid esters, triglycerides along with carbohydrate sources such as corn syrup, dextrins and glucose using a fermentation process comprising a wild-type or engineered microorganism. The production of sophorolipids with the use of renewable substrates and different microbial species, as well as the variation in culture parameters (e.g., incubation time, stirring speed, pH of the medium and added nutrients), allow for the acquisition of compounds with distinct structural and physical properties. This makes it possible to produce a wide variety of compounds that exhibit different physical, chemical, biochemical, and biophysical properties.

In preferred embodiments of the subject invention, the subject methods initially comprise producing standard sophorolipid molecules that are then used for producing modified sophorolipids. In certain embodiments, this entails cultivating a sophorolipid-producing yeast in a submerged fermentation reactor comprising a tailored oleochemical feedstock to produce a yeast culture product, said yeast culture product comprising fermentation broth, yeast cells and sophorolipids having a mixture of two or more molecular structures.

The mixture of molecular structures can comprise, for example, lactonic sophorolipids, linear sophorolipids, de-acetylated sophorolipids, mono-acetylated sophorolipids, di-acetylated sophorolipids, esterified sophorolipids, sophorolipids with varying hydrophobic chain lengths, sophorolipids with fatty acid-amino acid complexes attached, and others, including those that are and/or are not described within in this disclosure.

In certain embodiments, the distribution of the mixture of sophorolipids molecules can be altered by adjusting fermentation parameters, such as, for example, feedstock, fermentation time, and dissolved oxygen levels.

The sophorolipids according to the present invention can be derived via a fermentation process from a recombinant organism or by a strain that naturally produces sophorolipids. Non-limiting examples of sophorolipid-producing organisms include Candida bombicola, Candida apicola, Candida bogoriensis, Yarrowia lipolytica, Starmerella bombicola, Starmerella clade, Rhodotorula bogoriensis, Wickerhamiella domericqiae, and Wickerhamomyces anomalus. Some recombinant sophorolipid-producing microbes have been reported to allow control of sophorolipid structure. As a non-limiting example, certain recombinant S. bombicola strains may be utilized to produce either solely lactonic or solely acidic sophorolipids. In addition, a recombinant Candida bombicola strain with an acetyltransferase gene knockout can be used to produce sophorolipids without acetylation.

In preferred embodiments, the microorganism is a yeast or fungus. Examples of yeast and fungus species suitable for use according to the current invention, include, but are not limited to, Acaulospora, Aspergillus, Aureobasidium (e.g., A. pullulans), Blakeslea, Candida (e.g., C. albicans, C. apicola), Cryptococcus, Debaryomyces (e.g., D. hansenii), Entomophthora, Fusarium, Hanseniaspora (e.g., H. uvarum), Hansenula, Issatchenkia, Kluyveromyces, Mortierella, Mucor (e.g., M. piriformis), Meyerozyma (e.g., M. guilliermondii), Penicillium, Phythium, Phycomyces, Pichia (e.g., P. anomala, P. guilliermondii, P. occidentalis, P. kudriavzevii), Pseudozyma (e.g., P. aphidis), Rhizopus, Saccharomyces (S. cerevisiae, S. boulardii sequela, S. torula), Starmerella (e.g., S. bombicola), Torulopsis, Thraustochytrium, Trichoderma (e.g., T. reesei, T. harzianum, T. virens), Ustilago (e.g., U. maydis), Wickerhamomyces (e.g., W. anomalus), Williopsis, and Zygosaccharomyces (e.g., Z. bailii).

In preferred embodiments, the microorganisms are selected from, for example, Starmerella spp. yeasta and/or Candida spp. yeasta, e.g., Starmerella (Candida) bombicola, Candida apicola, Candida batistae, Candida floricola, Candida riodocensis, Candida stellate and Candida kuoi. In a specific embodiment, the microorganism is Starmerella bombicola, e.g., strain ATCC 22214.

In one embodiment, the fermentation reactor may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, samples may be taken from the vessel for enumeration, purity measurements, sophorolipid concentration, and/or visible oil level monitoring. For example, in one embodiment, sampling can occur every 24 hours.

The microbial inoculant according to the subject methods preferably comprises cells and/or propagules of the desired microorganism, which can be prepared using any known fermentation method. The inoculant can be pre-mixed with water and/or a liquid growth medium, if desired.

The microorganisms utilized according to the subject invention may be natural, or genetically modified microorganisms. For example, the microorganisms may be transformed with specific genes to exhibit specific characteristics. The microorganisms may also be mutants of a desired strain.

In one embodiment, a single type of microorganism is grown in a reactor system. In alternative embodiments, multiple microorganisms, which can be grown together without deleterious effects on growth or the resulting product, can be grown in a single reactor system. There may be, for example, 2 to 3 or more different microorganisms grown in a single reactor at the same time. In some embodiments, more than one microorganism grows symbiotically in the reactor.

In certain embodiments, the cultivation may be supplemented with a carbon source. The carbon source can be a carbohydrate, such as glucose, dextrose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as canola oil, soybean oil, rice bran oil, olive oil, corn oil, sunflower oil, sesame oil, and/or linseed oil; powdered molasses, etc. These carbon sources may be used independently or in a combination of two or more.

In certain embodiments, the cultivation may be supplemented with high-oleic acid and/or exclusively-oleic acid oleochemical feedstock, which results in a yeast culture product comprising a narrower diversity of sophorolipid molecular structures than with feedstocks containing sources of other fatty acids, wherein the principal sophorolipid molecules produced contain an 18C carbon chain and a: single unsaturated bond at the ninth carbon.

In one embodiment, the liquid growth medium comprises a nitrogen source. The nitrogen source can be, for example, yeast extract, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid growth medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These 20 inorganic salts may be used independently or in a combination of two or more.

In one embodiment, growth factors and trace nutrients for microorganisms are included in the medium. Inorganic nutrients, including trace elements such as iron, zinc, copper, manganese, molybdenum and/or cobalt may also be included in the medium. Furthermore, sources of vitamins, essential amino acids, proteins and microelements can be included, for example, corn flour, peptone, yeast extract, potato extract, beef extract, soybean extract, banana peel extract, and the like, or in purified forms. Amino acids such as, for example, those useful for biosynthesis of proteins, can also be included.

The method of cultivation can further provide oxygenation to the growing culture. One embodiment utilizes slow motion of air to remove low-oxygen containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid.

In one embodiment, the microorganisms can be grown on a solid or semi-solid substrate, such as, for example, corn, wheat, soybean, chickpeas, beans, oatmeal, pasta, rice, and/or flours or meals of any of these or other similar substances.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium chloride, and/or sodium carbonate. These inorganic salts may be used independently or in a combination of two or more.

In some embodiments, when, for example, the microbes used to inoculate the substrate are in spore form (e.g., bacterial endospores), germination enhancers can be added to the substrate. Examples of germination enhancers according to the present invention include, but are not limited to, L-alanine, manganese, L-valine, and L-asparagine or any other known germination enhancer.

The pH of the culture should be suitable for the microorganism of interest. Buffers, and pH regulators, such as carbonates and phosphates, may be used to stabilize pH near a preferred value. In certain embodiments, a base solution is used to adjust the pH of the culture to a favorable level, for example, a 15% to 30%, or a 20% to 25% NaOH solution. The base solution can be included in the growth medium and/or it can be fed into the fermentation reactor during cultivation to adjust the pH as needed. When metal ions are present in high concentrations, use of a chelating agent in the liquid medium may be necessary.

The method and equipment for cultivation of microorganisms and production of the microbial by-products can be performed in a batch, quasi-continuous, or continuous processes.

In one embodiment, the method of cultivation is carried out at about 5° to about 100 °C, about 15° to about 60 °C, about 20° to about 45 °C, about 22° to about 35 °C, or about 24° to about 28 °C. In one embodiment, the cultivation may be carried out continuously at a constant temperature. In another embodiment, the cultivation may be subject to changing temperatures.

According to the subject methods, the microorganisms can be cultivated in the fermentation system for a time period sufficient to achieve a desired effect, e.g., production of a desired amount of cell biomass or a desired amount of sophorolipids.

In certain embodiments, fermentation of the yeast culture occurs for about 40 to 150 hours, or about 48 to 140 hours, or about 72 to 130 hours or about 96 to 120 hours. In certain specific embodiments, fermentation time ranges from 48 to 72 hours, or from 96 to 120 hours.

In some embodiments, the sophorolipids produced by microorganisms of interest may be retained in the microorganisms or secreted into their growth medium. The sophorolipid content can be, for example, at least 20%, 30%, 40%, 50%, 60%, 70%, 80%, or 90%.

The growth medium may contain compounds that stabilize the activity of the sophorolipids. The sophorolipids can be purified, or the sophorolipids can be used in crude form, meaning they are not separated from the fermentation broth in which they were produced.

In certain embodiments, the sophorolipid is isolated and/or purified from the growth medium resulting from fermentation of a biosurfactant-producing microorganism. Isolation and purification can be easily achieved using standard methods or techniques described in the literature. The sophorolipid can be further concentrated, if desired.

### Modification of Sophorolipids

In one embodiment, the subject invention provides a synthetic procedure to create a sophorolipid scaffold to add modifications to the fatty acid end for enhancing dispersion ability. A “scaffold” is a starting point for creating a structure-activity relationship, e.g., determining how each small modification contributes to dispersion capabilities. For example, transforming the carboxylic acid into a methyl, ethyl, or isobutyl ester can be used to create dispersion agents out of fatty acid such as stearic acid.

In some embodiments, the modified sophorolipids according to the present invention are obtained by chemically modifying sophorolipids that have been obtained from the product of fermentation by sophorolipid-producing microbes. The sophorolipids to be chemically modified may be obtained by fermentation methods and processes as described above and/or any other methods known in the art. Any suitable techniques and chemical reactions known in the art may be used to modify sophorolipids.

Chemical modifications can also be made, for example, to alter the degree of unsaturation on the fatty acid chain. Commonly known hydrogenation or dehydrogenation reactions, or addition or elimination reactions may be used.

Sophorolipids containing an unsaturated bond at a specific position allows for site-directed functionalization of the sophorolipid molecule. In certain embodiments, the linear sophorolipids are ozonated with ozone gas. During ozonolysis of the linear sophorolipids, the olefin moiety of the sophorolipid molecule is converted to an ozonide, a reactive 5-membered ring. In preferred embodiments, the sophorolipid containing the ozonide is reduced to afford an aldehyde handle. In a specific embodiment, the reducing agent is triphenyl phosphine used in equimolar concentrations to the sophorolipid-ozonide.

In certain embodiments, the reactive aldehyde handle, whether produced via ozonolysis or oxidative cleavage, is then used as the site for addition of, for example, a primary amine via reductive amination. In certain embodiments, the reductive amination comprises introducing a primary amine to the sophorolipid-aldehyde under reducing conditions. This produces a stable secondary amine that serves as a covalent linkage between the sophorolipid scaffold and the cargo (e.g., amino acids) of the primary amine.

In certain embodiments, the reducing agent is sodium cyanoborohydride, sodium triacetoxyborohydride or sodium borohydride. In some embodiments, the linear sophorolipid aldehyde is extracted with ethyl acetate from the aqueous mixture and concentrated and dried under reduced pressure (e.g., about 200 to 250 mbar, or about 240 mbar) at a temperature of about 35 to 45 °C. The dried crude linear sophorolipid aldehyde can then be dissolved in a reaction medium comprising tetrahydrofuran (THF) and water. The percentage of water used as the reaction medium preferably does not exceed 50% water, and typically is from 0 to 25%.

In some embodiments, the amide coupling addition of amino acids such as phenylalanine methyl ester and tryptophan methyl ester could retain the biobased carbons of the amino acids while adding aromatic rings that retain dispersion-promoting properties of the sophorolipid. Biobased carbons are carbons directly derived from, for example, agricultural sources, e.g., sugar-based fermentation products. Biobased carbons can be quantitatively measured by C14 radiocarbon testing.

In one embodiment, modified sophorolipids of the present invention incorporate within their structure an amino acid linked to the fatty acid chain of the sophorolipid structure via the primary amine. By covalently attaching an amino acid to a sophorolipid or a truncated sophorolipid, the modified sophorolipids exhibit dispersion-promoting capabilities. Further, the addition of amino acids contributes biobased carbons instead of synthetic carbons, increasing the biobased carbon % in the modified sophorolipids, which enhances the dispersion properties of the modified sophorolipids. Biobased carbon % represents the number of biobased carbon out of the total number of atoms within a molecule (e.g., biobased carbon %=(the number of biobased carbons/total number of atoms)×100).

An “amino acid” as used herein has at least one amino group and at least one acid such as carboxyl group in its backbone, along with a side chain that is different for each amino acid. The side chain can be a variety of substituents, including but are not limited to, hydrogen (e.g., as in glycine), organic, semi-organic or inorganic, and a ring that is cyclized on to the amine (e.g., as in proline).

The amino acid as used herein may be L-amino acid or D-amino acid. It can be natural amino acids (such as essential amino acids, non-essential amino acids, or conditional amino acids) or synthetic/modified amino acids that do not exist in nature. The present invention encompasses amino acids that are both incorporated into proteins and not incorporated into proteins. Amino acids that exist in nature may be purified products or can be artificially synthesized.

In some embodiments, the amino acid according to the present invention is an alpha (α)-amino acid, which contains an amino group and a carboxyl group attached to the same carbon adjacent to the carboxyl group designated as the α-carbon. Non-limiting examples of α-amino acids include, for example, glycine, methionine, lysine, phenylalanine, tyrosine, tryptophan, and glutamine.

Other non-limiting examples of amino acids that can be used in the present invention include, but are not limited to, alanine, β-alanine, arginine, asparagine, aspartic acid, cysteine, homocysteine, glutamic acid, histidine, homotaurine, isoleucine, leucine, phenylalanine, proline, serine, threonine, tryptophan, tyrosine, valine, theanine, D-2-amino-3-guanidinopropionic acid, GABA, citrulline, tranexamic acid, aminocaproic acid, 4-amino-5-hexenoic acid, 4-oxaproline, 4-thioproline, 2-azaproline, 4-hydroxyproline, 1,5-disubstituted tetrazole, 2-amino isobutyric acid, sarcosine, 1-aminocyclopentane-1-carboxylic acid, beta alanine, 2-amino-cyclopentane carboxylic acid (beta-proline), 5-hydroxylysine, hydroxylysine-5-sulfate, hydroxylysine-5-nitrate, hydroxylysine-5-phosphate, serine-3-sulfate, threonine-3-sulfate, serine-3-nitrate, threonine-3-nitrate, serine-3-phosphate, threonine-3-phosphate, 2-hydroxy alkanoic acid, 5-aminopentanoic acid, and 5-amino-2-oxopentanoic acid.

In some embodiments, the modified sophorolipids having an attached amino acid can further be esterified by a functional group via the carboxyl group of the amino acid. The functional groups include, but are not limited to, for example, methyl, ethyl, propyl, butyl, pentyl, i-propyl, i-butyl, and

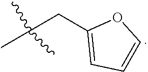 [SMILES: *Cc1ccco1]

Lactonic species inherently produce lower HLB values, so peracetylation or global transformation of sophorose hydroxyl groups to ethers would also lower the HLB value, creating biobased dispersion agents with high percentage of carbon (i.e., carbon %).

In one embodiment, the modified sophorolipids of the subject invention have a biobased carbon content, for example, from about 50% to about 100%, about 50% to about 95%, about 50% to about 90%, about 50% to about 80%, about 50% to about 70%, about 50% to about 60%, about 55% to about 90%, about 55% to about 85%, about 55% to about 80%, about 55% to about 75%, about 60% to about 85%, about 60% to about 80%, about 60% to about 75%, or about 60% to about 70%.

One embodiment is directed to a method for producing the modified sophorolipids comprising: obtaining the linear sophorolipid molecules; and functionalizing the linear sophorolipid molecules by converting all, or a majority, of the linear sophorolipid molecules to a modified sophorolipid molecule.

### Dispersion Formulations

The subject invention provides a method for dispersing a particulate substance in a liquid carrier, the method comprising contacting a dispersion agent with the particulate substance concurrently with, before or after mixing the particulate substance with the liquid carrier, wherein the dispersion agent comprises a biosurfactant. Advantageously, in preferred embodiments, the biobased dispersion agents are biodegradable and do not need to be removed from materials with which they are used, or the final products.

Preferably the dispersion agent comprises one or more of the modified sophorolipids of the present invention. The liquid may be an aqueous or non-aqueous fluid, including water, an alcohol, and/or an oil.

In one embodiment, the subject invention also provides a method for preventing or reducing the settling of a dispersed particulate substance in a liquid, wherein the method comprises applying the dispersion agent of the subject invention to the particulate substance and/or the liquid.

In certain embodiments, the dispersion agent adsorbs to the particulate substance and forms a repulsive barrier to interactive forces existing between the particles. For particles that have already formed agglomerates or aggregates, the dispersion agent can also penetrate between the particles to separate them from one another. The application of agitation or other shear forces can enhance the separation of particles.

In some embodiments, the dispersion agent provides stabilization of dispersions by adsorbing on the particles and altering the charge to the surface to promote repulsion from one another and/or from other components, such as carrier molecules.

Further provided herein are dispersion formulations comprising a dispersion agent according to the subject invention and a particulate substance in a carrier. The dispersion agent may be added at concentrations of at least, 0.001%, 0.005%, 0.01%, 0.05%, 0.1%, 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, or 70% by weight of the formulation.

In certain embodiments, the composition of the subject invention comprises the modified sophorolipid at, for example, about 0.001 to about 70%, about 0.01 to about 60%, about 0.1 to about 50%, about 0.1 to about 45%, about 0.1 to about 40%, about 0.1 to about 35%, about 0.1 to about 30%, about 0.1 to about 25%, about 0.1 to about 20%, about 0.1 to about 20%, about 0.1 to about 15%, about 0.1 to about 10%, about 0.1 to about 9.0%, about 0.1 to about 8.0%, about 0.1 to about 7.0%, about 0.1 to about 6.0%, about 0.1 to about 5.0%, about 0.1 to about 4.0%, about 0.1 to about 3.0%, about 0.1 to about 2.0%, about 1.0 to about 9.0%, about 1.0 to about 5.0%, about 1.0 to about 3.0%, about 3.0 to about 10%, about 3.0 to about 7.0%, about 5.0 to about 10%, about 5.0 to about 9.0%, about 6.0 to about 10%, about 7.0 to about 10%, about 8.0 to about 10%, about 5 to about 40%, about 10 to about 40%, about 10 to about 30%, about 10 to about 20%, about 20 to about 30%, about 20 to about 40%, about 20 to about 50%, about 30 to about 50%, or about 30 to about 40% by weight of the formulation.

In certain embodiments, the size of a biosurfactant micelle according to the subject invention is less than 10 nm, preferably less than 8 nm, more preferably less than 5 nm. In a specific embodiment, the size is from 0.8 nm to 1.5 nm, or about 1.0 to 1.2 nm. Advantageously, such small micelle size allows for enhanced penetration of biosurfactants into nanometer-sized spaces and pores, such as those between particles. This allows for enhanced adsorption onto the particles to improve dispersion.

The particulate substance can comprise particles ranging from, for example, 0.1 nanometer to 1 centimeter, about 1 nanometer to 1 millimeter, or about 5 nanometers to 1 micrometer in size, for example, a pigment or dye particle, a metallic particle, an organic particle, a polymeric particle, a liposomal particle, or others described herein. In some embodiments, the particulate substance is a nanoparticle having a particle size from about 0.1 to about 1,000 nanometers, about 1 to about 750 nanometers, about 1.5 to about 500 nanometers, about 2 to about 250 nanometers, about 2.5 to about 150 nanometers, or about 1 to 100 nanometers.

In some embodiments, the size of a particle refers to the diameter or approximate diameter of a particle. For a population of particles, this can also be referred to as a Z-average particle size, which can be measured according to routine protocols known to one skilled in the art.

In some embodiments, the size is measured by dynamic light scattering (DLS) (Z-average). In some embodiments, the size is measured by TEM (Transmission Electron Microscopy).

In some embodiments, the total amount of the particulate substance in the composition is from about 0.001 wt % to about 50 wt %, about 0.01 wt % to about 25 wt %, about 0.05 wt % to about 20 wt %, about 0.1 wt % to about 15 wt %, about 0.5 wt % to about 10 wt %, or about 1 wt % to about 5 wt % of the total composition.

Exemplary types of particulate substances according to the subject invention can include, for example, liposome-based particles, metallic particles, polymeric particles, organic particles, inorganic particles, viral particles, lipid-based particles, nanoparticle albumin-bound technology, quantum dots, nano-tubes, inorganic semiconductor nanocrystals, metallic nanospheres, and others known in the art.

In one embodiment, the dispersion formulation can comprise positively and negatively charged particles at a ratio of 1:10 to 10:1, positively charged to negatively charged.

In certain embodiments, the particle ingredient(s) can comprise, for example, particles and/or nanoparticles of: Aluminum Cerium Oxide; Aluminum Hydroxide; Aluminum Hydroxide Oxide; Aluminum; Aluminum; Aluminum Nitride; Aluminum Oxide; Aluminum Oxide, Silane Coated; Aluminum Titanate; Aluminum-doped Zinc Oxide; Antimony; Antimony Oxide; Antimony Tin Oxide (ATO); Arsenic Oxide; Barium Iron Oxide; Barium Oxide; Barium Strontium Titanate; Barium Sulfate; Barium Titanate; Barium Zirconate; Beryllium; Beryllium Oxide; Bismuth Cobalt Zinc Oxide; Bismuth; Bismuth Oxide; Boron Carbide; Boron; Boron Nitride; Boron Oxide; Cadmium Oxide; Calcium Carbonate; Calcium chloride; Calcium Hydrogen Phosphate; Calcium Oxide; Calcium Phosphate; Calcium Titanate; Calcium Zirconate; Carbon Black; Carbon; Carbon nanotubes; Cerium; Cerium Oxide, Calcium doped Nanopowder; Cerium Oxide, Gadolinium doped Nanopowder; Cerium Oxide, Samarium doped Nanopowder; Cerium Oxide, Yttria doped Nanopowder; Cerium Zirconium Oxide; Cesium Oxide; Chromium Carbide; Chromium Cobalt Iron; Chromium; Chromium Nitrate; Chromium Oxide; CIS; C-MITE Cerium Oxide; Cobalt Aluminum Oxide; Cobalt Iron; Cobalt Iron Oxide; Cobalt Iron Oxide; Cobalt Iron Zinc Oxide; Cobalt; Cobalt (II) Oxide; Cobalt (II,III) Oxide; Cobalt (III) Oxide; Copper Aluminum Oxide; Copper Indium Gallium Selenide; Copper Iron Oxide; Copper; Copper Nickel; Copper Oxide; Copper Tin Alloy; Copper Zinc Iron Oxide; Copper Zinc; Copper-Zinc Alloy; Diamond; Dysprosium; Dysprosium Oxide; Erbium; Europium; Europium Oxide; Ferrofluid; Fullerene Powder; Gadolinium; Gadolinium Oxide; Gallium Antimonide; Gallium Arsenide; Gallium Nitrate; Gallium Oxide; Gallium-doped Zinc Oxide; Germanium; Germanium Oxide; Gold; Gold on Carbon Black; Gold on Titania; Gold Oxide; Graphite; Hafnium; Hafnium Oxide; Holmium; Holmium Oxide; Hydroxyapatite; Indium Hydroxide; Indium; Indium Oxide; Indium Phosphide; Indium Tin Oxide; Iridium; Iridium Oxide; Iron Cobalt; Iron Hydroxide Oxide; Iron; Iron Nickel Copper; Iron Nickel; Iron Nickel Oxide; Iron (II,III) Oxide; Iron (III) Oxide; Lanthanum Hexaboride; Lanthanum; Lanthanum Nickelate; Lanthanum Nickelate, Strontium doped Nanopowder; Lanthanum Oxide; Lanthanum Strontium Manganese Oxide; Lanthanum Strontium Manganite; Lanthanum Trifluoride; Lead; Lead Oxide; Lithium Carbonate; Lithium Cobalt Oxide; Lithium Iron Phosphate; Lithium Manganese Oxide; Lithium; Lithium Oxide; Lithium Titanate; Lithium Titanate Spinel; Lithium Vanadate; Lutetium; Lutetium Oxide; Magnesium Aluminate, Spinel; Magnesium Aluminum Oxide; Magnesium Hydroxide; Magnesium Iron Oxide; Magnesium; Magnesium Oxide; Magnesium Zinc Iron Oxide; Manganese Iron Oxide; Manganese; Manganese Oxide; Manganese Titanium Oxide; Manganese Zinc Iron Oxide; Molybdenum Carbide; Molybdenum; Molybdenum Oxide; Molybdenum Sulfide; Neodymium; Neodymium Oxide; Nickel Chromium Oxide; Nickel Cobalt Chromium; Nickel Cobalt Iron Oxide; Nickel Cobalt Oxide; Nickel Hydroxide; Nickel; Nickel Oxyhydroxide; Nickel Titanium; Nickel Zinc Iron Oxide; Nickel (II) Oxide; Nickel (III) Oxide; Niobium Boride; Niobium Carbide; Niobium; Niobium Nitride; Niobium Oxide; Osmium; Osmium Oxide; Palladium; Palladium Entrapped in Aluminum Hydroxide Matrix; Palladium Oxide; Platinum; Platinum on Carbon Black; Platinum on Titania; Platinum Oxide; Potassium Oxide; Praseodymium; Praseodymium Oxide; Rhenium; Rhenium Oxide; Rhodium Entrapped in Aluminum Hydroxide Matrix; Rhodium Oxide; Rubidium Oxide; Ruthenium; Ruthenium Oxide; Samarium; Samarium Oxide; Samarium Strontium Cobalt Oxide; Scandium; Scandium Oxide; Selenium; Selenium Oxide; Silica; Silicon Aluminum; Silicon Carbide; Silicon Carbide Nitride; Ruthenium Oxide; Silicon; Silicon Nitride; Silicon Oxide; Silver Copper; Silver; Silver Oxide; Silver Platinum; Silver Tin Alloy; Sodium Oxide; Stainless Steel; Strontium Aluminum Oxide; Strontium Carbonate; Strontium Ferrite; Strontium Iron Oxide; Strontium; Strontium Oxide; Strontium Titanate; Sulfur; Tantalum Carbide; Tantalum; Tantalum Oxide; Tellurium; Tellurium Oxide; Terbium; Terbium Oxide; Thallium; Thallium Oxide; Thorium Oxide; Thulium; Thulium Oxide; Tin; Tin Oxide; Tin Silver Copper; Titanium Boride; Titanium Boride-Boron Carbide; Titanium Boride-Boron Carbide-Tungsten Boride; Titanium Boron Oxide; Titanium Boride; Titanium Carbide; Titanium Carbon; Titanium Carbon Nitrate; Titanium Carbonitride; Titanium; Titanium Nitride; Titanium Oxide; Anatase; Titanium Dioxide, Rutile; Titanium Silicate; Titanium (IV) Oxide, Mixture of Rutile and Anatase; Tungsten Carbide-Cobalt; Tungsten Carbide; Tungsten Disulfide; Tungsten; Tungsten Oxide; Tungsten Sulfide; Vanadium Carbide; Vanadium; Vanadium Nitride; Vanadium Oxide; Ytterbium Fluoride; Ytterbium; Ytterbium Oxide; Yttria Stabilized Zirconia; Yttria Stabilized Zirconia; Yttrium Aluminate; Yttrium Aluminum Oxide; Yttrium Europium Oxide; Yttrium Iron Oxide; Yttrium; Yttrium Oxide; Zinc Iron Oxide; Zinc; Zinc Oxide; Zinc Titanate; Zirconium Carbide; Zirconium Hydroxide; Zirconium; Zirconium Nitrate; Zirconium Oxide; and Zirconium (IV) Silicate;

Carbon Black; Carbon Electrodes; Carbon Fabric; Carbon Fiber; Carbon Foam; Carbon Granules; Carbon Nanoparticles; Carbon Nanorods; Carbon Nanotube Ink; Carbon Nanotubes; Carbon Pieces; Carbon Powder; Carbon Slugs; Copper Carbon Nanotubes; Double-Walled Carbon Nanotubes; Graphene; 3D Graphene Foam; Graphene Monolayer; Graphene Multilayer; Graphene Nanoplatelets; Graphene Oxide Monolayer; Graphene Oxide Paper; Graphene Oxide Thin Film; Graphite Nanofibers; Graphite Nanopowder; Graphite Paste; Graphite Powder; Graphite Precipitate; Graphite Rod; Graphite Shavings; Graphite, Expandable; Graphite, Fluorinated, Polymer; Graphite, Micronized; Graphite, Natural Amorphous; Graphite, Natural Flake; Graphite, Spherical; Lithium Titanate; Manganese Selenide; Mesoporous Carbon; Multi-Walled Carbon Nanotubes; Potassium Graphite; Pyrolytic Graphite; and Tunable Nanoporous Carbon;

Coating materials, such as, for example, epoxies; polyvinyl chlorides; polyurethanes; polysiloxanes; alkyds; resins; rubber; and acrylics;

Non-limiting examples of inorganic pigment particles include purple pigments: Ultramarine violet (PV15; Na₆Al₆Si₆O₂₄S₄), Han Purple (BaCuSi₂O₆), Cobalt Violet (PV14; Co3 (PO₄)2), and Manganese violet (PV16; NH₄MnP₂O₇);
blue pigments: Ultramarine blue (PB29; Na₆Al₆Si₆O₂₄S₄), Cobalt Blue (PB28) and Cerulean Blue (PB35) cobalt(II) stannate, Egyptian Blue (CaCuSi₄O₁₀), Han Blue (BaCuSi₄O₁₀), Azurite (Cu₃(CO₃)2(OH)₂), Prussian Blue (PB27; Fe₇(CN)₁₈), YlnMn Blue (Yl-xMnxO₃), and selected copper phtalocyanines;
green pigments: Chrome green (PG17; Cr₂O₃), Viridian (PG18; Cr₂O₃·H₂O), Cobalt green or Rinman's green or Zinc green (CoZnO₂), Malachite (Cu₂CO₃(OH)₂), Paris Green (Cu(C₂H₃O₂)₂-3Cu(AsO₂)₂), Scheele's Green or Schloss Green (CuHAsO₃), Verdigris (Cu(CH₃CO₂)₂), selected copper phtalocyanines, and Green earth (K [(Al,FeIII), (FeII,Mg](AlSi₃,Si4)O₁₀(OH)₂);
yellow pigments: aureolin or Cobalt Yellow (PY40; K₃Co(NO₂)₆), Yellow Ochre (PY43; Fe₂O₃·H₂O), Titanium Yellow (PY53; NiO·Sb₂O₃: 20TiO₂), and Mosaic gold (SnS₂);
red pigments: Sanguine, Caput Mortuum, Indian Red, Venetian Red, Oxide Red (PR102; iron oxides), Red Ochre (PR102; anhydrous Fe₂O₃), Burnt Sienna (PBr7; anhydrous Fe₂O₃);
brown pigments: Raw Umber (PBr7; Fe₂O₃+MnO₂+nH₂O+Si+AlO₃+), and Raw Sienna (PBr7; limonite clay);
black pigments: Carbon Black (PBk7), Ivory Black (PBk9), Vine Black (PBk8), Lamp Black (PBk6), Mars Black or Iron black (PBk11; Fe₃O₄), manganese dioxide (MnO₂), and titanium (III) oxide (Ti₂O₃);
white pigments: stibous oxide (Sb₂O₃), barium sulphate (BaSO₄), lithopone (BaSO₄*ZnS), titanium dioxide (TiO₂), and zinc oxide (ZnO); and
organic pigments including but not limited to, azo pigments, phthalocyanines, quiacridone, diaryl pyrrolopyrroles, lithol, toluidine derivatives, pyrazolones, dinitroaniline, Hansa yellow, indanthrenes, dioxazine and benzimidazolone.

In some embodiments, the particulate substance is a natural or synthetic dye. Natural dyes can be derived from plant, fungal, mineral and/or animal sources. Non-limiting examples of natural dyes and/or sources of natural dyes include cochineal, lac, urine, murex snail, octopus/cuttlefish, cutch tree, gamboge tree resin, chestnut, rhubarb, Indigofera, kamala seed, madder root, mangosteen, myrobalan, pomegranate, teak leaf, weld, black walnut, sumac tree, Acer sp., Pinus edulis, Rhus trilobata, lupine, Phoradendron juniperinum, Marsdenia, Polygonum tinctorum, Lonchocarpus cyanescens, Acacia spp., kermes, Brazilwood, Lithospermum purpurocaeruleum, mulberry, Genista tinctoria, woad, fustic, iron, corn husk, Artemisia tridentate, red onion, tyrian, saffron, pomegranate, turmeric, safflower, onionskins, weld, quercitron, fustic, butternut, yellow root, Rumex crispus, snake weed, rubber plant, rabbitbush, rose hip, juniper, alder, henna, alkanet, asafetida, sappanwood, Rubia spp., Sarcodon squamosus, Hydnellum geogenium, Hypholoma fasciculare, Phaeolus schgeinitzii, Pisolithus tinctorius, Rocella tinctoria, cudbear, archil, litmus, crottle, wine, grapes, cactus fruit, tea, coffee and blood.

Synthetic dyes can include, but are not limited to, acid or anionic dyes, basic or cationic dyes, azoic or naphthol dyes, direct dyes, disperse dyes, reactive dyes, sulfur dyes, vat dyes, anthraquinones, phthalocyanines and triarylmethanes.

In some embodiments, the ingredient in need of dispersion is a plant, animal, fungus or mineral ingredient for the formulation of food, beverages and/or supplements.

In some embodiments, the ingredient in need of dispersion is a vitamin and/or mineral for example, vitamins A, E, K3, D3, B1, B3, B6, B12, C, biotin, folic acid, panthothenic acid, nicotinic acid, choline chloride, inositol and para-amino-benzoic acid, calcium, magnesium, phosphorus, potassium, sodium, chlorine, sulfur, chromium, cobalt, copper, iodine, iron, manganese, molybdenum, nickel, selenium, zinc, antioxidants, beta-glucans, bile salt, cholesterol, enzymes, carotenoids, and many others.

In some embodiments, the ingredient in need of dispersion is an oil-based ingredient, such as an essential oil for use in a cleaning product or cosmetic product.

The dispersion formulations of the subject invention comprising the modified sophorolipid(s) can be useful in, for example, paints, coatings, printing, packaging, plastics and composites, electronics, oil and gas fluids, food processing, wastewater processing, agriculture, pharmaceuticals, textile production, pulp and paper processing, concrete and stucco, cleaning compositions and cosmetics. Advantageously, the present invention can be used without causing harm to users and without releasing large quantities of polluting and toxic compounds into the environment.

The formulations may further include additional active or inactive ingredients, suitable for the mode of administration and intended purpose, provided that such addition does not adversely interfere with the functions of the modified sophorolipids.

In some embodiments, the composition comprises additional biosurfactants, for example, glycolipids, lipopeptides, flavolipids, phospholipids, fatty acid esters, and high-molecular-weight biopolymers such as lipoproteins, lipopolysaccharide-protein complexes, and/or polysaccharide-protein-fatty acid complexes.

In one embodiment, the additional biosurfactant is a glycolipid, such as, for example, rhamnolipids (RLP), cellobiose lipids, trehalose lipids and/or mannosylerythritol lipids (MEL). In one embodiment, the biosurfactant is a lipopeptide, such as, for example, surfactin, iturin, fengycin, arthrofactin, amphisin, viscosin, lichenysin, paenibacterin, polymyxin and/or battacin. In certain embodiments, the glycolipid is a rhamnolipid. Rhamnolipids comprise a glycosyl head group (i.e., a rhamnose) moiety, and a 3-(hydroxyalkanoyloxy)alkanoic acid (HAA) fatty acid tail, such as, e.g., 3-hydroxydecanoic acid. Two main subtypes of rhamnolipids exist, mono- and di-rhamnolipids, which comprise one or two rhamnose moieties, respectively. The HAA moiety can vary in length and degree of branching, depending on, for example, the growth medium and the environmental conditions. The highest accumulation of rhamnolipids (RLP) has been shown by submerged cultivation of Pseudomonas spp., such as P. aeruginosa.

Rhamnolipids according to the subject invention can have the following structure, according to General Formula (3):

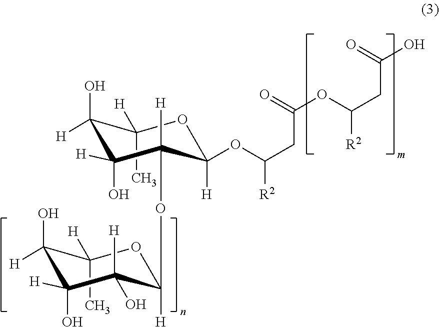

wherein m is 2, 1 or 0,
n is 1 or 0,
R¹ and R² are, independently of one another, the same or a different organic functional group having 2 to 24, preferably 5 to 13 carbon atoms, in particular a substituted or unsubstituted, branched or unbranched alkyl functional group, which can also be unsaturated,
wherein the alkyl functional group is a linear saturated alkyl functional group having 8 to 12 carbon atoms, or is a nonyl or a decyl functional group or a mixture thereof.

Salts of these compounds are also included according to the invention. In the present invention, the term “di-rhamnolipid” is understood to mean compounds of the above formula or the salts thereof in which n is 1. Accordingly, “mono-rhamnolipid” is understood in the present invention to mean compounds of the general formula or the salts thereof in which n is 0. In certain specific embodiments, the composition comprises a mixture of mono- and di-rhamnolipids.

In some embodiments, a mixture of modified and natural sophorolipids are incorporated into the dispersion formulation. In certain embodiments, the total biosurfactant content of the subject invention ranges from about 0.0001% to 99.9% wt %, 0.001% to 90%, 0.01% to 85%, 0.015% to 80%, 0.1% to 75%, 0.15% to 70%, 0.2% to 65%, 0.25% to 60%, 0.3% to 55%, 0.35% to 50%, 0.4% to 45%, 0.45% to 40%, 0.5% to 35%, 0.55% to 30%, 0.6% to 25%, or 0.65% to 20% wt %.

Non-biological surfactants can also be added to the composition. Examples of surfactants include, but are not limited to, alkyl sulfates, alkyl ether sulfates (e.g., sodium/ammonium lauryl sulfates and sodium/ammonium laureth sulfates), amphoterics (e.g., amphoacetates and amphopropionates), sulfosuccinates, alkyl polyglucosides, betaines (e.g., cocamidopropyl betaine), sultaines, sacrosinates, isethionates, taurates, ethoxylated sorbitan esters, alcohol ethoxylates, alkanolamides and amino acid-based surfactants.

Other active dispersion agents, such as polyacrylates, polyethylene oxides, polypropylene oxides, and copolymers containing them may be used in combination with the biobased dispersion agent according to the invention.

Other optional components can include but are not limited to carriers (e.g., water), binders, solvents, thickeners, detergents, corrosion inhibitors, biocides, UV protectants, crystal modifiers, stabilizers, antioxidants, fragrances, defoamers, emulsifiers, preservatives, pH adjusters, and buffering agents, which are added in amounts effective to perform their intended function. Identification and use of these additives, and amounts thereof, is well within the skill of the art.

### Uses for Dispersion Formulations

In some embodiments, methods are provided for improving paints and coatings, wherein the paint and/or coating is formulated with a dispersing agent of the subject invention therein. In certain embodiments, the paint and/or coating comprises one or more particulate ingredients, such as pigments, UV-resisting agents, antimicrobials and/or anti-corrosion agents. Advantageously, when the paint/coating formulation is applied to a surface, the particulate ingredient(s), such as pigments, can be more uniformly spread over the surface due to the presence of the dispersing agent, thus providing greater overall coverage of the surface and/or protection of the surface from, for example, corrosion, microorganisms, and/or UV rays.

In certain embodiments, the paint/coating formulation can comprise, in addition to a pigment and the dispersing agent, a carrier (e.g., water or oil), binders, resins, solvents, co-solvents, viscosity modifiers, thickeners, additional surfactants, plasticizers, pH modifiers, buffers, preservatives, biocides, defoamers and other ingredients known in the art.

Binders, which are primarily responsible for adhesion of the coating to an object, can include, for example, acrylic, alkyds, acrylic acid, acrylamide, phenolic, phenolic-alkyd, polyacrylamide, polyurethanes, silicone-alkyd, polyesters, epoxies, vinyl, vinyl acetate-ethylene, vinyl-alkyd, inorganic binders (sodium, potassium ethyl silicate, lithium, etc.), organic binders (carbon-based), Tectyl® (Daubert Chemical Company, Inc., Chicago, IL), aliphatic-urethanes, and oil-modified urethanes.

Solvents can include, for example, mineral or organic solvents, including, for example, ethanol, butanol, propanol, aliphatic hydrocarbons, alicyclic hydrocarbons, xylene, toluene, d-limonene, ketones, and/or isopropyl alcohol. In certain embodiments, the paint/coating formulation comprises water as a solvent. The water can be filtered by granular-activated carbon, deionized, distilled, or processed by reverse osmosis.

Additionally, pH modifiers can be used to increase or decrease the pH to, preferably, facilitate the dissolution of various components of the formulation. The water-based formulations can be acrylic-based or latex-based. The latex can be from a natural origin, such as, for example, a flowering plant (angiosperm), or, preferably, the latex is synthetically derived from, for example, polymerizing styrene. The acrylic base for a paint/coating can be created from acrylic resins, which are synthetic thermoplastics.

In certain embodiments, the paint/coating can be oil-based. Synthetic or natural resins can be used in combination with any one of the aforementioned solvents to create the oil-based resin. Alkyd resins can be, for example, used in the subject composition. Alkyd resins can be created using natural oils, such as, for example, linseed oil, safflower oil, soybean oil, sunflower oil, tung oil, or castor oil.

In certain embodiments, the paint/coating can comprise corrosion inhibitors, such as, for example, zinc oxide, 2-aminomethyl propanol, diethylethanolamine benzotraizole, and methyl benzotriazole.

The paint/coating compositions of the subject invention can be applied to a variety of inorganic or organic objects such as, for example, steel, aluminum, wood, plastic, gypsum, paper, silk, glass, cotton, concrete, plaster, clay, stucco, plastic, rubber, hair, skin, fur, or plants. The compositions can be applied to objects that reside a range of temperatures, aquatic environments, or other stress-inducing conditions.

In certain embodiments, the dispersion formulations of the subject invention can also have applications in the oil and gas industry. For example, in some embodiments, the dispersion agent can be utilized for enhancing the dispersion of nano-proppants in fracturing fluids, which can be injected into a formation to prop nanoscale fractures produced by hydraulic fracturing. In some embodiments, the nano-proppant comprises, for example, carbon black, carbon nanotubes and nanofibers, fumed silica and alumina and/or cellulosic nanofibers, nanoclays and finely divided grades of fly ash. In certain embodiments, the dispersion agent can also be formulated into injection fluids for enhancing oil recovery from a well by, for example, penetrating and dispersing deposits such as paraffins, asphaltenes, scales and biofilms; and penetrating rock pores and dispersing oil into water-based injection fluids.

In certain embodiments, the subject invention provides a method for dispersing particles in a liquid from various sources, including, for example, mining sites or quarrying sites.

In some embodiments, the dispersion formulations described herein may be used for water treatment, including industrial, municipal, agricultural, food processing, and oilfield water treatment. Specifically, the dispersion agent described herein may be added to water to enhance penetration of organic and inorganic residue, chemicals, FOG, deposits and biofilms to loosen and disperse them for further treatment and/or filtering.

In certain embodiments, the compositions of the subject invention can be used for agricultural applications. Environmentally-conscious nanofertilizers can provide efficient ion and nutrient delivery into plant cells. See Miranda-Villagomez et al. 2019, incorporated herein by reference in its entirety.

In certain embodiments, the dispersion agent described herein can be formulated a nanofertilizer component. Nanofertilizers can comprise, for example, nanoscale fertilizers (nanoparticles that contain nutrients), nanoscale additives (traditional fertilizers with nanoscale additives) and nanoscale coatings (traditional fertilizers coated or loaded with nanoparticles). In some embodiments, the nanoparticle ingredient is a nanoparticle containing one or more nutrients, such as, for example, carbon (C), hydrogen (H), oxygen (O), nitrogen (N), phosphorus (P), potassium (K), sulfur (S), calcium (Ca), magnesium (Mg), boron (B), chlorine (Cl), copper (Cu), iron (Fe), manganese (Mn), molybdenum (Mo), nickel (Ni), and zinc (Zn). In certain embodiment, a nanoparticle loaded with the one or more nutrients is utilized, for example, a chitosan, polyacrylamide, polyacrylate, or zeolite nanoparticle.

Advantageously, the agricultural dispersion formulation can enhance dispersion of nutrients within soil and enhance nutrient availability and absorption by plant roots compared with the application of nanoparticle ingredient(s) (e.g., nanofertilizers) without the dispersion agent.

### Methods of Dispersing in Mining

In certain embodiments, the subject dispersant compositions can be applied directly to mined particles, including, for example, minerals, metals, elements, or compounds. The subject compositions can be used in methods to disperse mined particles in a liquid and inhibit the settling of the particles. In other embodiments, the dispersant compositions can be applied to the vessel that can contain the mined particle. The vessel can be, for example, a barrel, silo, storage tank, pipe, drain, pump, impeller, tube, holding pond, flotation cell, or bucket.

In certain embodiments, the addition of a dispersant composition to a liquid (e.g., water) containing mined particles can reduce the interfacial tension between water and the particles.

In certain embodiments, the subject invention provides a method for mineral flotation from a mining site, which involves crushing ore, adding water and the subject dispersant composition to the ore particles, and then floating the mineral of interest to the surface of the water.

In certain embodiments, the mining site can include a coal mine, an iron ore mine (e.g., taconite), copper mine, copper-nickel mine, tin mine, nickel mine, gold mine, silver mine, molybdenum mine, aluminum mine (e.g., bauxite mine, kyanite mine), lead-zinc mine, tungsten mine, phosphate mine, potash mine, mica mine, bentonite mine, or zinc mine. The mine can be an underground mine, surface mine, placer mine or in situ mine.

In certain embodiments, the dispersant composition can be sprayed or poured on to mined particles and mixed with the mined particles to disperse the particles throughout a liquid, including, for example, water. In certain embodiments, the biosurfactants of the subject compositions can inhibit particle agglomeration by reducing the interfacial tension. In certain embodiments, the dispersant composition is used with agitation to reduce the agglomeration of particles.

In some embodiments, the subject dispersant composition can entirely replace or partially replace (e.g., reduce the use of) conventional chemical surfactants in methods of dispersing mined particles.

In certain embodiments, the time period in which the dispersant composition can be contacted to the liquid and/or mined particle is about 1 second to about 1 year, about 1 minute to about 1 year, about 1 minute to about 6 months, about 1 minute to about 1 month, about 1 minute to about 1 week, about 1 minute to about 48 hours, about 30 minutes to about 40 hours, or preferably about 12 hours to about 24 hours.

In certain embodiments, the amount of dispersant composition applied is about 0.00001 to 15%, about 0.00001 to 10%, about 0.0001 to 5%, about 0.001 to 3%, about 0.01%, or about 1 vol % based on an amount of liquid that is treated. In certain embodiments, the concentration and rate of application of the dispersant composition is dependent on the volume of liquid being treated.

In certain embodiments, the methods of the subject invention result in at least a 25% decrease in the amount of agglomerated particles when compared to a suspension without the use of a dispersant, preferably at least a 50% decrease, after one treatment. In certain embodiments, a liquid composition containing a mined particle can be treated multiple times to further decrease the amount of agglomerated particles.

In certain embodiments, the dispersant composition according to the subject invention is effective due to reducing the adhesion of particles. In some embodiments, the sophorolipid, rhamnolipid, or other biosurfactant serves as a vehicle for facilitating dispersal of particles in a liquid.

Examples of target metals that can be dispersed using the methods of the subject invention, as well as ores and/or minerals that produce and/or comprise the target metals, include but are not limited to cobalt (e.g., erythrite, skytterudite, cobaltite, carrollite, linnaeite, and asbolite (asbolane)); copper (e.g., chalcopyrite, chalcocite, bornite, djurleite, malachite, azurite, chrysocolla, cuprite, tenorite, native copper and brochantite); gold (e.g., native gold, electrum, tellurides, calaverite, sylvanite and petzite); silver (e.g., sulfide argentite, sulfide acanthite, native silver, sulfosalts, pyrargyrite, proustite, cerargyrite, tetrahedrites); aluminum (e.g, bauxite, gibbsite, bohmeite, diaspore); antimony (e.g., stibnite); barium (e.g., barite, witherite); cesium (e.g., pollucite); chromium (e.g., chromite); cadmium (e.g., sphalerite, greenockite, hawleyite, ramdohrite); iron (e.g., hematite, magnetite, pyrite, pyrrhotite, goethite, siderite); lead (e.g., galena, cerussite, anglesite); lithium (e.g., pegmatite, spodumene, lepidolite, petalite, amblygonite, lithium carbonate); magnesium (e.g., dolomite, magnesite, brucite, carnallite, olivine); manganese (e.g., hausmannite, pyrolusite, barunite, manganite, rhodochrosite); mercury (e.g., cinnabar); molybdenum (e.g., molybdenite); nickel (e.g., pentlandite, pyrrhotite, garnierite); phosphorus (e.g., hydroxylapatite, fluorapatite, chlorapatite); platinum group (platinum, osmium, rhodium, ruthenium, palladium) (e.g., native elements or alloys of platinum group members, sperrylite); potassium (e.g., sylvite, langbeinite); rare earth elements (cerium, dysprosium, erbium, europium, gadolinium, holmium, lanthanium, lutetium, neodymium, praseodymium, samarium, scandium, terbium, thulium, ytterbium, yttrium) (e.g., bastnasite, monazite, loparite); sodium (e.g., halite, soda ash); strontium (e.g., celestite, strontianite); sulfur (e.g., native sulfur, pyrite); tin (e.g., cassiterite); titanium (e.g., scheelite, huebnerite-ferberite); uranium (e.g., uraninite, pitchblende, coffinite, carnotite, autunite); vanadium; zinc (e.g., sphalerite, zinc sulfide, smithsonite, hemimorphite); and zirconium (e.g., zircon).

Additional elements and/or minerals, the dispersion of which the subject invention is useful, include, e.g., arsenic, bertrandite, bismuthinite, borax, colemanite, kernite, ulexite, sphalerite, halite, gallium, germanium, hafnium, indium, iodine, columbite, tantalite-columbite, rubidium, quartz, diamonds, garnets (almandine, pyrope and andradite), corundum, barite, calcite, clays, feldspars (e.g., orthoclase, microcline, albite); gemstones (e.g., diamonds, rubies, sapphires, emeralds, aquamarine, kunzite); gypsum; perlite; sodium carbonate; zeolites; chabazite; clinoptilolite; mordenite; wollastonite; vermiculite; talc; pyrophyllite; graphite; kyanite; andalusite; muscovite; phlogopite; menatite; magnetite; dolomite; ilmenite; wolframite; beryllium; tellurium; bismuth; technetium; potash; rock salt; sodium chloride; sodium sulfate; nahcolite; niobium; tantalum and any combination of such substances or compounds containing such substances.

All patents, patent applications, provisional applications, and publications referred to or cited herein are incorporated by reference in their entirety, including all figures and tables, to the extent they are not inconsistent with the explicit teachings of this specification.

### REFERENCES

Miranda-Villagómez, Iris, L., Gómez-Merino, Fernando Carlos, S.-V., Manuel, Sánchez-García, & Ángel, M. (2019 Nov. 18). Nanophosphorus Fertilizer Stimulates Growth and Photosynthetic Activity and Improves P Status in Rice.
Fairhurst, D., 2013. “An Overview of the Zeta Potential-Part 1: The Concept.” American Pharmaceutical Review. https://www.americanpharmaceuticalreview.com/Featured-Articles/133232-An-Overview-of-the-Zeta-Potential-Part-1-The-Concept/(Accessed Mar. 22, 2021).
Fairhurst, D., 2013. “An Overview of the Zeta Potential-Part 2: Measurement.” American Pharmaceutical Review. https://www.americanpharmaceuticalreview.com/Featured-Articles/134634-An-Overview-of-the-Zeta-Potential-Part-2-Measurement/(Accessed Mar. 22, 2021).
Fairhurst, D., 2013. “An Overview of the Zeta Potential-Part 3: Uses and Applications.” American Pharmaceutical Review. https://www.americanpharmaceuticalreview.com/Featured-Articles/139288-An-Overview-of-the-Zeta-Potential-Part-3-Uses-and-Applications/(Accessed Mar. 22, 2021).

1. A compound having a structure selected from:
a) General Formula (II): 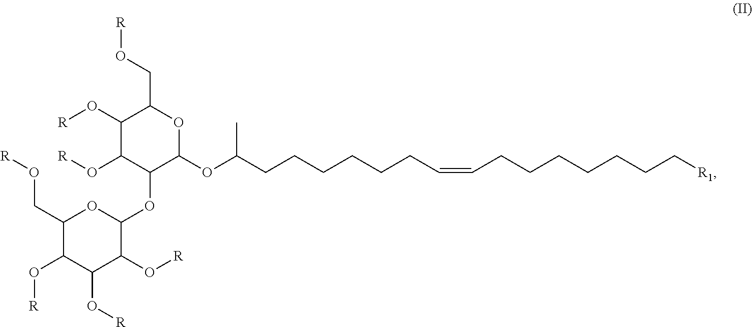
b) General Formula (III) 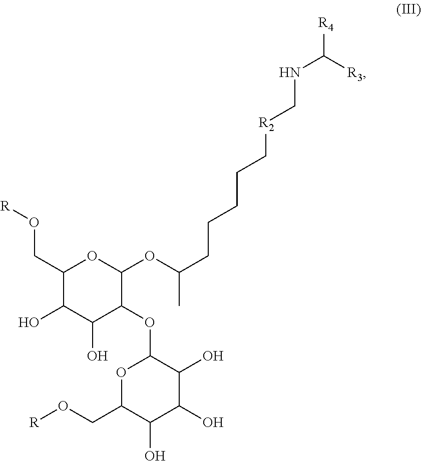
c) General Formula (IV) 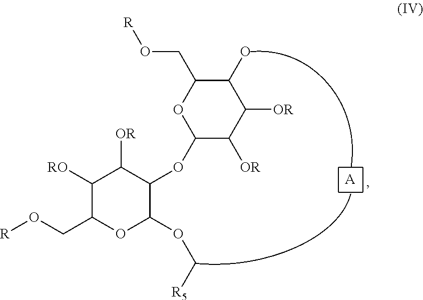
d) General Formula (V) 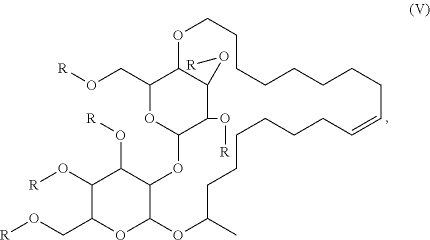
e) General Formula (VI) 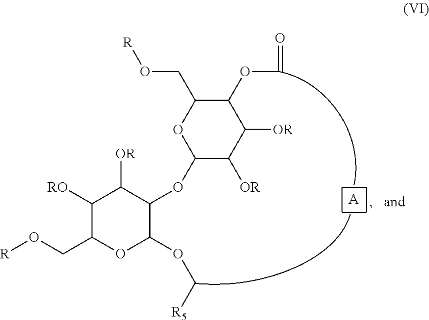
f) General Formula (VII) 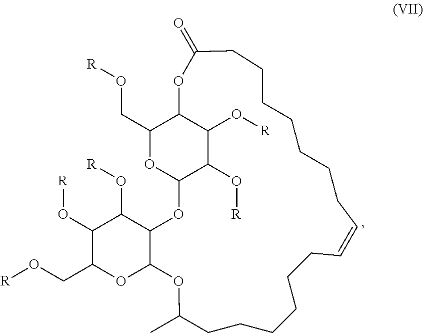
wherein each R is independently selected from the following: 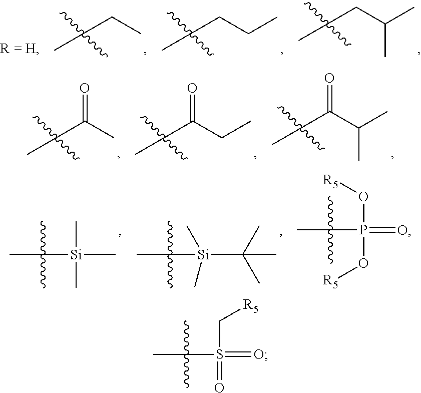
wherein each R₁ is independently selected from the following: 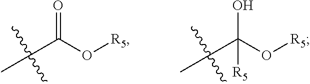
wherein each R₂ is independently selected from the following: CH₂, (CH₂)₂, (CH₂)₃, (CH₂)₄, (CH₂)₅, (CH₂)₆, (CH₂)₇, (CH₂)₈, (CH₂)₉ and (CH₂)₁₀; wherein each R₃ is independently selected from the following: H, methyl, ethyl, propyl, butyl, pentyl, i-propyl, i-butyl, 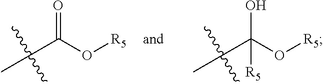
wherein each R₄ is independently selected from the following: 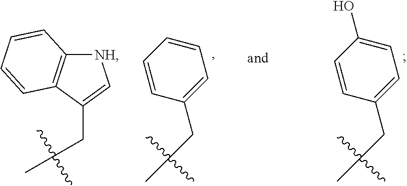
wherein each R₅ is independently selected from the following: H, methyl, ethyl, propyl, butyl, pentyl, i-propyl, i-butyl, and 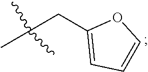 [SMILES: *Cc1ccco1]
and
wherein A is an aliphatic chain with 10-21 carbons comprising zero, one, two or three unsaturations.

2. The compound of claim 1, which has a hydrophile-lipophile balance (HLB) value ≤6.

3. The compound of claim 1, which have a biobased carbon content of from about 50% to about 80%.

4. The compound of claim 1, which have a biobased carbon content of from about 60% to about 75%.

5. A dispersion formulation comprising the compound of any of claims 1-4, a particulate substance and a carrier.

6. The dispersion formulation of claim 5, wherein the particulate substance has a particle size from 0.1 nm to 1 cm.

7. The dispersion formulation of claim 5, wherein the carrier is water, an alcohol or an oil.

8. The dispersion formulation of claim 5, wherein the particulate substance is a pigment or dye. 2

9. A paint composition comprising the compound of claim 1, and one or more of the following additional components: a pigment, a dye, water, a solvent, an additional biosurfactant, an additional surfactant, a binder, a preservative, a thickener, a resin, a buffer, a pH modifier, a UV resisting agent, a biocide, a corrosion inhibitor and/or a viscosifier.

10. A method for dispersing a particulate substance in a liquid, the method comprising applying a dispersion agent of claim 1 to the particulate substance and/or to the liquid.

11. The method of claim 10, further comprising measuring the degree of dispersion of the particulate substance in the liquid.

12. A method for stabilizing a dispersion formulation comprising a particulate substance dispersed in a liquid, the method comprising applying a dispersion agent of claim 1 to the dispersion formulation.

13. A method for dispersing a solid mineral in a liquid, the method comprising applying a composition comprising a biosurfactant to the solid mineral and/or to the liquid.

14. The method of claim 13, further comprising measuring the degree of dispersion of the solid mineral in the liquid.

15. The method of claim 13, wherein the solid mineral is from a coal mine, iron ore mine, copper mine, copper-nickel mine, tin mine, nickel mine, gold mine, silver mine, molybdenum mine, aluminum mine, lead-zinc mine, tungsten mine, phosphate mine, potash mine, mica mine, bentonite mine, zinc mine, or any combination thereof.

16. The method of claim 13, wherein the biosurfactant is a glycolipid and/or a yeast culture comprising a glycolipid.

17. The method of claim 16, wherein the yeast culture is a Starmerella sp. and/or a Candida sp. yeast.

18. The method of claim 16, wherein the glycolipid is a sophorolipid, mannosylerythritol lipid, trehalose lipid, rhamnolipid, or any combination thereof.

19. The method of claim 18, wherein the sophorolipid is a linear sophorolipid or a lactonic sophorolipid.

20. The method of claim 13, further comprising agitating the composition and the solid mineral and/or the liquid.
